(19) United States
(12) Patent Application Publication
(10) Pub. No.: US 2022/0114213 A1
(43) Pub. Date: Apr. 14, 2022

(54) SYSTEM AND METHOD FOR CLINICAL DECISION SUPPORT FOR THERAPY PLANNING USING CASE-BASED REASONING

(71) Applicant: KONINKLIJKE PHILIPS N.V., EINDHOVEN (NL)

(72) Inventors: LILLA BOROCZKY, MOUNT KISCO, NY (US); MARK R. SIMPSON, WHITE PLAINS, NY (US); YE XU, HARTSDALE, NY (US); MICHAEL CHUN-CHIEH LEE, BRONX, NY (US)

(21) Appl. No.: 17/515,635

(22) Filed: Nov. 1, 2021

Related U.S. Application Data
(63) Continuation of application No. 13/993,419, filed on Jun. 12, 2013, now abandoned.
(60) Provisional application No. 61/423,801, filed on Dec. 16, 2010.

(51) Int. Cl. G06F 16/90; G16H 50/70

(52) U.S. Cl. CPC G06F 16/90; G16H 50/70

(57) ABSTRACT

A non-transitory computer-readable storage medium storing a set of instructions executable by a processor. The set of instructions is operable to receive a current patient set of data relating to a current patient; compare the current patient set of data to a plurality of previous patient sets of data, each of the previous patient sets of data corresponding to a previous patient; select one of the previous patient sets of data based on a level of similarity between the selected previous patient set of data and the current patient set of data; and provide the selected previous patient set of data to a user.

### BACKGROUND

A doctor planning a course of treatment for a patient may typically have a variety of treatment options available for selection. Each treatment option may have various advantages and disadvantages and may affect the patient's future prognosis in varying ways. The advantages and disadvantages of a given possible course of treatment may depend on various characteristics of the patient. A doctor may wish to research treatments and results for prior similar patients before making a treatment decision for the current patient.

### SUMMARY OF THE INVENTION

A non-transitory computer-readable storage medium stores a set of instructions executable by a processor. The set of instructions is operable to receive a current patient set of data relating to a current patient; compare the current patient set of data to a plurality of previous patient sets of data, each of the previous patient sets of data corresponding to a previous patient; select one of the previous patient sets of data based on a level of similarity between the selected previous patient set of data and the current patient set of data; and provide the selected previous patient set of data to a user.

A system includes a user interface, a database, and a similarity search mechanism. The user interface receives a current patient set of data relating to a current patient. The database stores a plurality of previous patient sets of data. Each of the previous patient sets of data corresponds to a previous patient. The similarity search mechanism searches the plurality of previous patient sets of data and selecting one of the previous patient sets of data having a high degree of similarity to the current patient set of data. The selected one of the previous patient sets of data is provided to the user by the user interface.

### BRIEF DESCRIPTION OF THE DRAWINGS

FIG. 1 illustrates a system for providing case-based decision support according to an exemplary embodiment.

FIG. 2 illustrates a first method for providing case-based decision support according to an exemplary embodiment.

FIG. 3 illustrates an exemplary graphical user interface for providing results of a method such as the method of FIG. 2 to a user.

FIG. 4 illustrates a second method for providing case-based decision support according to an exemplary embodiment.

FIG. 5 illustrates a third method for providing case-based decision support according to an exemplary embodiment.

### DETAILED DESCRIPTION

The exemplary embodiments may be further understood with reference to the following description and the appended drawings, wherein like elements are referred to with the same reference numerals. The exemplary embodiments describe systems and methods by which case-based reasoning is applied to provide decision support for doctors making treatment decisions for patients.

When a patient is diagnosed with an illness or other condition, a doctor (or other medical professional) must determine a course of treatment appropriate to the patient's condition. Decisions made during this process are based on a variety of factors. These factors include the nature and details of the patient's illness, the patient's medical history, the patient's family history, any existing co-morbidities, other medications currently being administered to the patient, the patient's preferences such as quality-of-life preferences, etc. The doctor may base such decisions in part on knowledge in the field, which includes experiences with previous patients having similar conditions, treatments administered to those previous patients, and the outcomes experienced by the previous patients after receiving treatment. While an individual doctor has his or her own past experiences available to draw on in the course of making such decisions, it may be desirable to have a broader array of information available to doctors in this situation. The exemplary embodiments provide doctors with access to information about a large number of previous patients in order to provide better treatment.

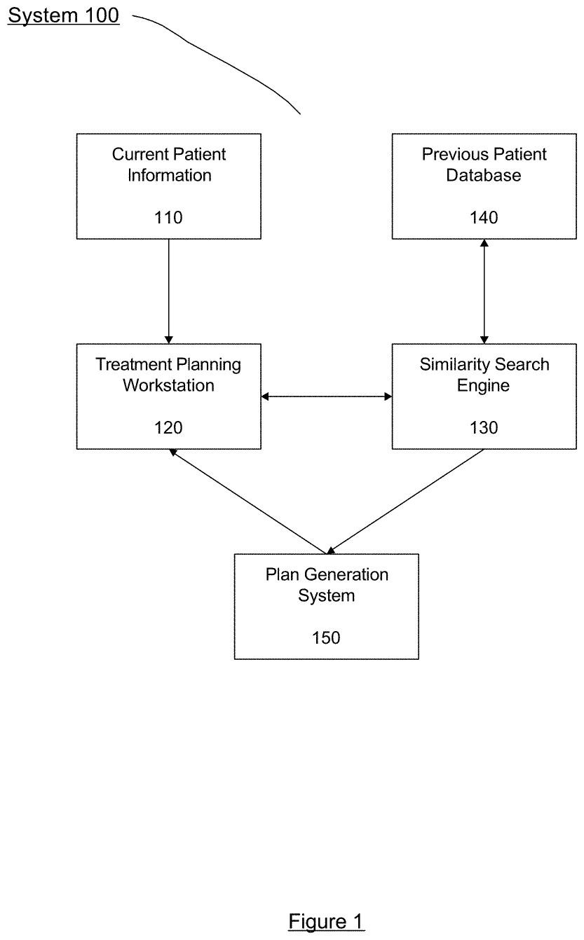

FIG. 1 illustrates a schematic view of an exemplary system 100. The lines connecting the elements shown in FIG. 1 may be any type of communications pathway suitable for conveying data between the elements so connected; arrows on the lines indicate the direction of data flow between the elements. The system 100 includes current patient information 110, which may be obtained, in various implementations, using any method for obtaining information about a patient that is known in the art. This may include an apparatus for generating medical images (e.g., a CT scanner, an X-ray imager, an MRI imager, etc.), the input of data provided by the patient (e.g., symptoms, medical history, etc.), etc.

For example, in the case of a newly-diagnosed breast cancer patient, the current patient information 110 typically includes one or more of demographics (e.g., age, height, weight, etc.), specifics of the diagnosis such as pathology results related to cancer type (e.g., infiltrating lobular carcinoma, ductal carcinoma in-situ (DCIS)), cancer subtypes (e.g., ER+/−, PR+/−, HER2+/−), staging of the cancer, co-morbidities (e.g., diabetes, high blood pressure, etc.), family history, and factors relating to quality of life. Typically, the current patient information 110 is available digitally, such as via one or more of a Hospital Information System (HIS), a Laboratory Information System (LIS), a Radiology Information System (RIS), a Picture Archiving and Communications System (PACS), and a Digital Pathology (DP) Information Management System.

The current patient information 110 is provided to a treatment planning workstation 120, which is a computing system (e.g., a combination of hardware and software) used by a doctor or other medical professional to plan treatment for the current patient. The treatment planning workstation 120 is similar to known systems presently used by medical professionals, except as will be described hereinafter.

The treatment planning workstation 120 transmits current patient information to a similarity search engine 130. The similarity search engine 130 also retrieves data on previous patients from a previous patient database 140, which is then compared to the information about the current patient as will be described in further detail hereinafter. The previous patient database 140 stores information in a repository using known medical informatics standards such as DICOM or DICOM-RT, but the data may also be stored using any other appropriate system. Data stored for previous patients may include medical images (e.g., x-ray, CT, MRI, etc.), prior patient medical history, treatment administered to the prior patient, prior patient outcomes (e.g., time of survival, time to progression, etc.). Additionally, the information stored in the previous patient database 140 for each patient may include further relevant information such as age, patient's family medical history, further information about the patient's current condition, other treatment currently being administered to the patient (e.g., chemotherapy), or any other information that may be relevant for the doctor to plan a course of treatment for the current patient.

Some or all of the data relating to previous patients is then transmitted from the similarity search engine 130 to a plan generation system 150, which generates a plan of treatment for the current patient based on the data relating to previous patients, as will be described in further detail hereinafter. The plan generation system 150 is also coupled with the treatment planning workstation 120, in order that its output may be returned to the planner who is using the treatment planning workstation. Those of skill in the art will understand that the similarity search engine 130, the previous patient database 140, and the plan generation system 150 may be implemented in various ways, including as hardware and/or software elements of the treatment planning workstation 120, or as separate hardware and/or software components, without impacting their functions. For example, previous patient database 140 can be embodied as any form of known hierarchical or relational database stored on any type of known computer-readable storage device. Plan generation system 150 and search engine 130 can be embodied as any standard computing system having computer-readable instruction processing and information storage hardware and software features.

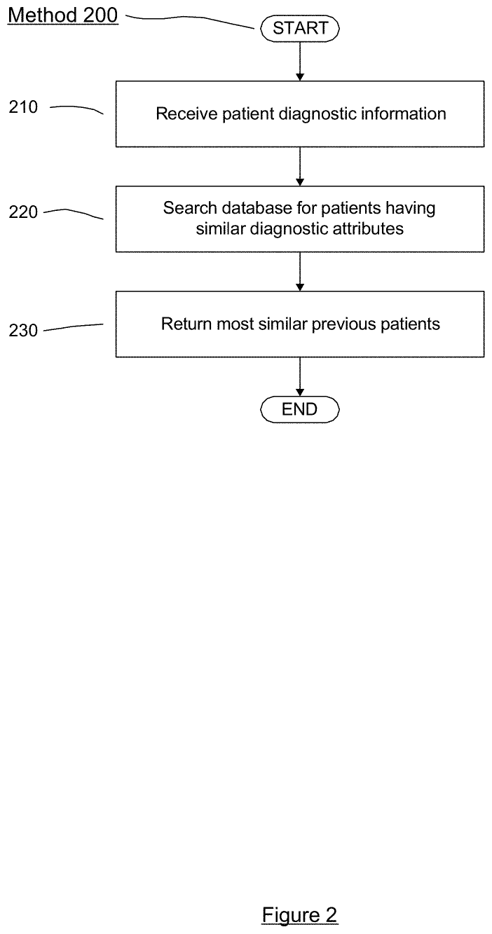

FIG. 2 illustrates an exemplary method 200 for retrieving data on previous patients having characteristics similar to the current patient, which will be described herein with reference to the exemplary system 100 of FIG. 1. In step 210, the current patient information 110 is received; as described above, this may be obtained by any means of obtaining such information as is known in the art. For example, the current patient information 110 is generated contemporaneously with the performance of the exemplary method 200 (e.g., medical images taken at this time); in another alternative situation, the current patient information 110 may have been generated previously, and may be stored in any suitable manner (e.g., in hardcopy, in a computer database, etc.). In another alternative situation, the patient's doctor may narrow the current patient information 110 to a relevant subset of all information that is available at this stage. The current patient information 110 (or a relevant subset thereof) is transmitted from the treatment planning workstation 120 to the similarity search engine 130.

In step 220, the similarity search engine 130 searches the previous patient database 140, using the current patient information 110 (or a relevant subset thereof), to find similar previous patients, i.e., previous patients whose characteristics (e.g., age, condition, medical history, etc.) are similar to the current patient.

When the search is proceeding in step 220, the current patient and the previous patients are represented as a set of features, each of which is an individual characteristic of the patients. A feature may be, for example, any of the characteristics discussed above with reference to the current patient information, e.g., cancer type. Features that are qualitative are represented as binary values; for example, if a feature under consideration is diabetes, the feature may be assigned a value of 0 if the current patient does not have diabetes or a value of 1 if the current patient has diabetes. Features that have more than one possible value may be represented on the same scale; for example, if a patient has a type of lesion that can have four different shapes, the feature corresponding to that lesion could be assigned to have a predetermined value of 0.25, 0.50, 0.75 or 1 depending on the shape of the lesion.

In addition to features that are directly measured or observed, some features may be computer-calculated, such as by the treatment planning workstation 120. For example, where the current patient information 110 includes medical images (e.g., MRI images), computer-calculated features may include a location of a cancerous lesion, its location relative to other organs, its size, shape, and margin, the size and assessment of the patient's lymph nodes, kinetic assessment of contrast uptake, etc., that may be determined based on the medical images. Some of this information may be determined through known image processing/analysis techniques such as image segmentation, image contouring, and other measurement tools for example, or other types of computer assisted diagnosis (“CAD”) tools.

For one exemplary search including K number of features, each feature may be identified by a feature index k ranging from 1 to K, and each feature may have a weight wₖ representing the weight to be given to that particular feature in the comparison. As one example, the sum of all weight values wₖ is equal to 1. The similarity between the current patient and any given previous patient may be represented as a “distance metric” based on the difference between each of the features, and based on the feature weights. The distance metric may be calculated based on a Euclidean distance, a city block distance, a Mahalanobis distance, or any other metric suitable for such calculation. In one exemplary embodiment, the distance metric between the current patient i and a previous patient j is calculated as:

Dᵢⱼ=ΣΣwₖ(f_clinicalₖᵢ)²+ΣΣwₖ(f_calculatedₖᵢ−f_calculatedₖⱼ)²+ΣΣwₖ(f_qualitylifeₖᵢ−f_qualitylifeₖⱼ)²+ΣΣwₖ(f_treatmentₖᵢ−f_treatmentₖᵢ)²

In the above expression, f_clinical represents features based on the patient's clinical information, f_calculated represents computer-calculated features for a patient, f_qualitylife represents quality-of-life related features for a patient, and f_treatment represents features related to a treatment plan for a patient. Quality-of-life features may include, for example, the patient's ability to perform his or her job, the patient's ability to take care of his or her family, whether the patient's treatment requires inpatient or outpatient care, etc. In the exemplary method 200, the search is based on the patient's clinical information, calculated features, and quality-of-life factors; therefore, the above expression may be simplified as:

Dᵢⱼ=ΣΣwₖ(f_clinicalₖᵢ)²+ΣΣwₖ(f_calculatedₖᵢ−f_calculatedₖⱼ)²+ΣΣwₖ(f_qualitylifeₖᵢ−f_qualitylifeₖⱼ)²

In step 230, previous patients having low distance metrics (i.e., a high degree of similarity to the current patient) are returned from the previous patient database 140 and provided to the doctor via the treatment planning workstation 120. As one example, the previous patients are shown using a visual representation of the previous patients and their degree of similarity to the current patient. This may be indicated using a histogram, a spider graph, or in various other manners known in the art.

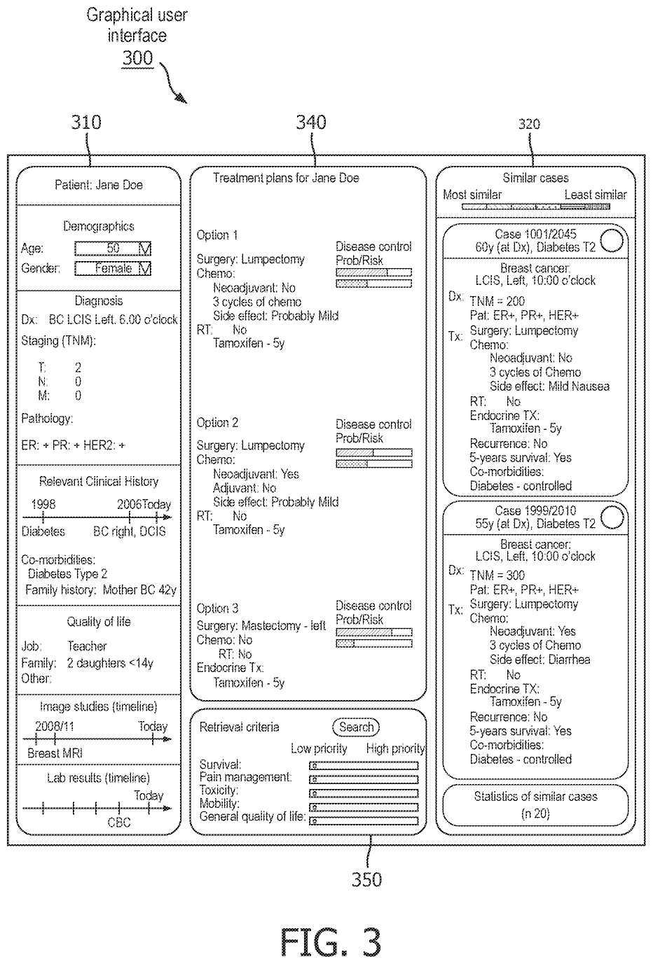

FIG. 3 illustrates an exemplary graphical user interface 300 by which results may be presented to a doctor (e.g., on a display of the treatment planning workstation 120). The graphical user interface 300 includes current patient information 310; the specific information shown may be customizable by the user (e.g., doctor). In the exemplary graphical user interface 310 of FIG. 3, the current patient information 310 includes name, age, gender, diagnosis, clinical history, co-morbidities, relevant family history, quality of life issues, a timeline of medical images, and a timeline of lab results. Those of skill in the art will understand that the specific information provided about the current patient may vary among differing embodiments.

The graphical user interface 300 also includes previous patient information 320. The previous patient information 320 includes relevant information about similar previous patients that are the results of a search such as that in step 230 of exemplary method 200. In the exemplary graphical user interface 300 of FIG. 3, two previous patients are shown, and the information provided about each previous patient includes a reference identifier, age, diagnosis, treatment administered, co-morbidities, and outcomes (e.g., recurrence, 5-year survival). Each previous patient listing may be accompanied by an indication of the degree of similarity between the previous patient and the current patient; in the exemplary embodiment, an indicator may be shown in a color ranging from green (representing a highest level of similarity) to red (representing a lowest level of similarity), but those of skill in the art will understand that other types of indications, such as a numerical representation or a graphical representation, are possible. Further, those of skill in the art will understand that the number of previous patients simultaneously shown, and the specific information shown about each previous patient, may vary among differing embodiments.

The graphical user interface 300 also includes retrieval criteria 330, which may be used by the doctor to weight various factors to be used in the search processes described above with reference to method 200 and below with reference to methods 400 and 500. For example, a doctor who desires a high degree of weight to be placed on pain management may configure the retrieval criteria 330 to reflect this preference.

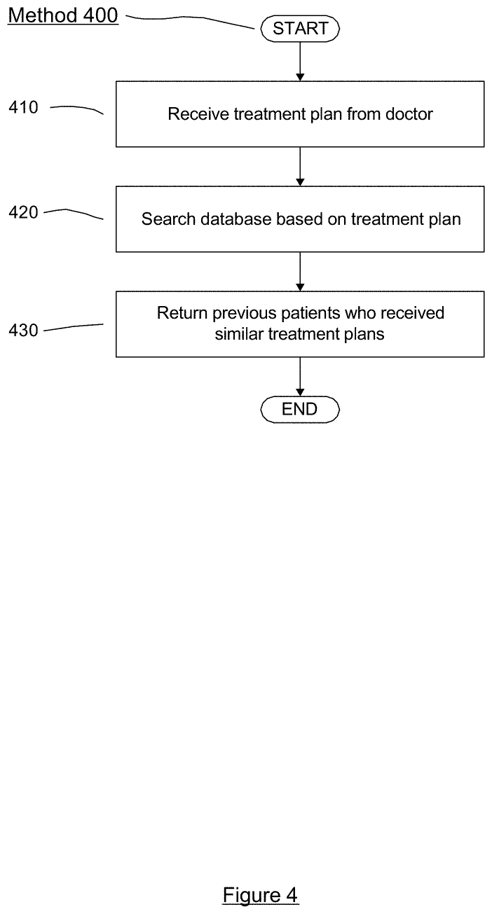

FIG. 4 illustrates a second exemplary method 400 for case-based decision support. The method 400 will be described with reference to the exemplary system 100 of FIG. 1. In step 410, a treatment plan for a current patient is received from a doctor; the treatment plan is based on the doctor's education and experience and the knowledge of the patient's symptoms, medical history, etc. A treatment plan may include a type of medication to be administered, a type of surgery to be performed, etc. The treatment plan is entered by the doctor (or, alternately, by a member of support staff) using treatment planning workstation 120.

In step 420, the similarity search engine 130 searches the previous patient database 140 for patients that have undergone treatment plans similar to the treatment plan that was entered in step 410. This step is substantially similar to step 220 of method 200, except that the features to be used in the search are features relating to the proposed treatment plan rather than features relating to the patient's diagnostic and other relevant clinical information. Elements of a treatment plan may be converted into features suitable for searching in the same manner described above. The distance metric between two patients for a search based on treatment plan-related features is expressed as:

Dᵢⱼ=ΣΣwₖ(f_treatmentₖᵢ−f_treatmentₖⱼ)²

In step 430, patients having low distance metrics (e.g., a high level of similarity to the current patient) are returned and provided to the doctor via the treatment planning workstation 120. As one example, the previous patients are shown using a visual representation of the previous patients and their degree of similarity to the current patient; this may be accomplished using a graphical user interface 300 as described above.

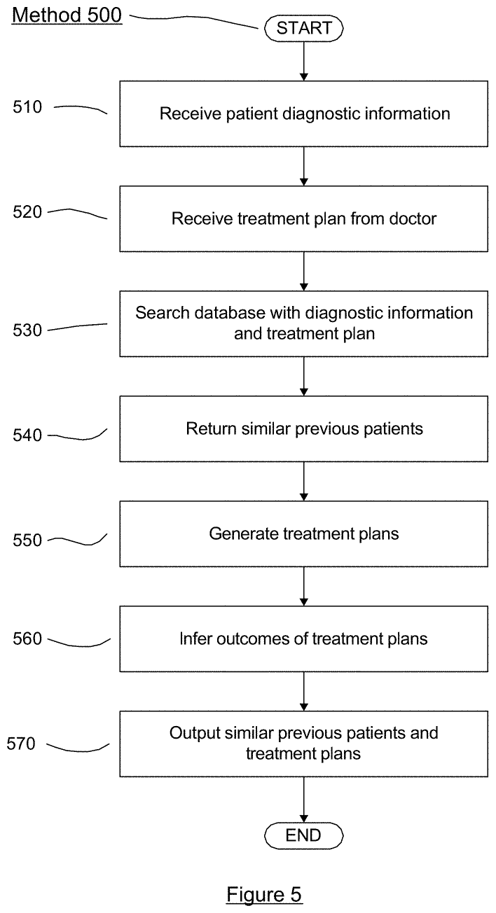

FIG. 5 illustrates a third exemplary method 500 for case-based decision support. In step 510, patient diagnostic information is received, as described above with reference to step 210 of method 200. In step 520, a treatment plan for the patient is received, as described above with reference to step 410 of method 400. In step 530, the similarity search engine 130 searches the previous patient database 140 using all received inputs as search criteria; this step may use all search parameters, as exemplified by the expression:

Dᵢⱼ=ΣΣwₖ(f_clinicalₖᵢ)²+ΣΣwₖ(f_calculatedₖᵢ−f_calculatedₖⱼ)²+ΣΣwₖ(f_qualitylifeₖᵢ−f_qualitylifeₖⱼ)²+ΣΣwₖ(f_treatmentₖᵢ−f_treatmentₖᵢ)²

In step 540, the search of step 530 results in the return of previous patients having a high degree of similarity to the current patient, as determined by a low distance score as expressed above. In step 550, one or more proposed treatment plans for the current patient are generated by the plan generation system 150 based on the treatment plans that were previously administered to one or more patients having a high degree of similarity to the current patient. In one instance, a treatment plan identical to that of the most similar previous patient (e.g., the previous patient with the lowest distance score) is proposed for the current patient. Alternatively, a treatment plan is determined based on a weighted average of similar patients. In such an example, the number of similar patients to be used may be predetermined, may be user-configurable, or may be a weighted average of all previous patients or all previous patients having the same condition as the current patient. The previous patients are typically weighted based on their level of similarity to the current patient, with patients having a higher level of similarity to the current patient weighted more heavily.

As another alternative example, an initial treatment plan is defined based on key differences between the characteristics of the current patient and those of previous patients. This approach may be valuable because, even in a large database, it may not be possible to find a perfect match for the current patient. Thus, in such an instance, the current patient is compared to a most similar previous patient, or a group of most similar previous patients. A key difference (or a number of differences) between the previous patient or patients and the current patient are identified, and treatment plan elements that are heavily dependent on that difference are determined based on knowledge in the field. A separate search is then conducted, based on the key difference, to find the closest patient who shares the key difference with the current patient, and the plan element relating to the key difference is taken from the patient found by that search. For example, high blood pressure is an important factor in determining a chemotherapy regimen for a patient. Thus, if the current patient has high blood pressure, and the most similar previous patient did not have high blood pressure, a separate search is conducted to find the most similar previous patient who did have high blood pressure, and the chemotherapy regimen for the current patient is based on the most similar previous patient with high blood pressure.

In another exemplary situation, the plan generation system 150 generates a plurality of treatment plans for the current patient. These may each be the treatment plan of an individual previous patient, or may be based on varying search criteria (e.g., weighting quality of life factors more or less heavily in the search). In step 560, the plan generation system 150 infers expected outcomes relating to each of the treatment plans if each of the treatment plans were to be administered to the current patient. The expected outcomes may be based on the outcomes experienced by previous patients who underwent similar treatment plans, the characteristics of the current patient, the manner in which the characteristics of the current patient differ from those of previous patients, etc. In step 570, the similar previous patients, treatment plans, and inferred outcomes are provided to the doctor using the graphical user interface 300 of the treatment planning workstation 120. FIG. 3 illustrates an embodiment showing three proposed treatment plans 340 for the current patient.

The exemplary embodiments described herein enable a doctor to consider a greater knowledge base of information in determining a treatment plan for a current patient than the doctor, as an individual, possesses. The exemplary embodiments further aid in the generation of a treatment plan for the current patient that is of a greater quality than one that is created by the doctor on an ad hoc basis based on the doctor's own experience. Further, because of the objective nature of the comparison to past patients, the quality of care received by patients may be standardized, rather then dependent upon the skills and experience of the doctor. Additionally, because proposed treatment plans for the current patient are based on one or more previous patients sharing characteristics with the current patient, higher quality treatment plans may be automatically generated for consideration by a treating doctor.

Those skilled in the art will understand that the above-described exemplary embodiments may be implemented in any number of manners, including, as a separate software module, as a combination of hardware and software, etc. For example, the similarity search engine 130 may be a program containing lines of code that, when compiled, may be executed on a processor.

It is noted that the claims may include reference signs/numerals in accordance with PCT Rule 6.2(b). However, the present claims should not be considered to be limited to the exemplary embodiments corresponding to the reference signs/numerals.

It will be apparent to those skilled in the art that various modifications may be made in the present invention, without departing from the spirit or the scope of the invention. Thus, it is intended that the present invention cover modifications and variations of this invention provided they come within the scope of the appended claims and their equivalents.

1. A computer-aided diagnostic system comprising:
a first interface to a database of prior patient records,
wherein each prior patient record comprises a plurality of prior patient features that are each assigned a non-binary numerical value,
wherein a substantial majority of the prior patient records are unrelated to prior patient records that are associated with a user of the system;
a second interface to a current patient record associated with the user,
wherein the current patient record comprises a plurality of current patient features corresponding to the plurality of prior patient features,
wherein each current patient feature is assigned a non-binary numerical value;
a processor; and
a display;
wherein for each prior patient record:
for each prior patient feature, the processor determines a numerical distance between the prior patient feature and the corresponding current patient feature;
wherein the processor determines a composite distance measure associated with each prior patient record based on the numerical distances of each prior patient feature and a non-zero weighting factor associated with each prior patient feature;
wherein the processor identifies a first prior patient record and a second prior patient record based on the composite distance measures of each prior patient record;
wherein the processor generates a method of treatment plan,
wherein a first element of the method of treatment plan is copied from a first treatment plan identified in the first prior patient record,
wherein a second element of the method of treatment plan is copied from a second treatment plan identified in the second prior patient record;
wherein the second element is an element that is:
related to a feature of the current patient that differs from a corresponding feature in the first prior patient record, and
related to a feature of the current patient that is similar to a corresponding feature in the second prior patient record;
wherein the processor presents the method of treatment plan to the user on the display.

2. The system of claim 1, wherein the database comprises hundreds of prior patient records.

3. The system of claim 2, wherein each prior patient record comprises at least ten prior patient features that are each assigned a non-binary numerical value.

4. The system of claim 1, wherein the composite distance measure is a Mahalanobis distance.

5. The system of claim 1, wherein the composite distance measure is one of: a Euclidean distance and a city block distance.

6. The system of claim 1, wherein:
each prior patient record is associated with a prior patient,
each prior patient record also includes prior patient information comprising at least one of: clinical information about the previous patient, calculated information about the previous patient, treatment plans of the previous patient, and outcome information of the previous patient,
the current patient record also includes current patient information comprising at least one of: clinical information about the current patient, calculated information about the current patient, treatment plans of the current patient, and outcome information of the current patient, and
the composite distance measure is further based on the current and prior patient information.

7. A computer-aided diagnostic method comprising:
accessing a database of prior patient records,
wherein each prior patient record comprises a plurality of prior patient features that are each assigned a non-binary numerical value,
wherein a substantial majority of the prior patient records are unrelated to prior patient records that are associated with a user of the system;
accessing a current patient record associated with the user,
wherein the current patient record comprises a plurality of current patient features corresponding to the plurality of prior patient features,
wherein each current patient feature is assigned a non-binary numerical value;
using a processor to determine a method of treatment plan based on the current patient record and the prior patient records, and
presenting the method of treatment plan to the user;
wherein for each prior patient record:
for each prior patient feature, the processor determines a numerical distance between the prior patient feature and the corresponding current patient feature;
wherein the processor determines a composite distance measure associated with each prior patient record based on the numerical distances of each prior patient feature and a non-zero weighting factor associated with each prior patient feature;
wherein the processor identifies a first prior patient record and a second prior patient record based on the composite distance measures of each prior patient record;
wherein a first element of the method of treatment plan is copied from a first treatment plan identified in the first prior patient record,
wherein a second element of the method of treatment plan is copied from a second treatment plan identified in the second prior patient record;
wherein the second element is an element that is:
related to a feature of the current patient that differs from a corresponding feature in the first prior patient record, and
related to a feature of the current patient that is similar to a corresponding feature in the second prior patient record.

8. The method of claim 7, wherein the database comprises hundreds of prior patient records.

9. The method of claim 8, wherein each prior patient record comprises at least ten prior patient features that are each assigned a non-binary numerical value.

10. The method of claim 7, wherein the composite distance measure is a Mahalanobis distance.

11. The method of claim 7, wherein the composite distance measure is one of: a Euclidean distance and a city block distance.

12. The method of claim 7, wherein:
each prior patient record is associated with a prior patient,
each prior patient record also includes prior patient information comprising at least one of: clinical information about the previous patient, calculated information about the previous patient, treatment plans of the previous patient, and outcome information of the previous patient,
the current patient record also includes current patient information comprising at least one of: clinical information about the current patient, calculated information about the current patient, treatment plans of the current patient, and outcome information of the current patient, and
the composite distance measure is further based on the current and prior patient information.

13. A non-transitory computer-readable medium that includes a program that, when executed by a processor, causes the processor to:
access a database of prior patient records,
wherein each prior patient record comprises a plurality of prior patient features that are each assigned a non-binary numerical value,
wherein a substantial majority of the prior patient records are unrelated to prior patient records that are associated with a user of the system;
access a current patient record associated with the user,
wherein the current patient record comprises a plurality of current patient features corresponding to the plurality of prior patient features,
wherein each current patient feature is assigned a non-binary numerical value;
determine a method of treatment plan based on the current patient record and the prior patient records;
present the method of treatment plan to the user;
wherein for each prior patient record:
for each prior patient feature, the processor determines a numerical distance between the prior patient feature and the corresponding current patient feature;
wherein the processor determines a composite distance measure associated with each prior patient record based on the numerical distances of each prior patient feature and a non-zero weighting factor associated with each prior patient feature;
wherein the processor identifies a first prior patient record and a second prior patient record based on the composite distance measures of each prior patient record;
wherein a first element of the method of treatment plan is copied from a first treatment plan identified in the first prior patient record,
wherein a second element of the method of treatment plan is copied from a second treatment plan identified in the second prior patient record;
wherein the second element is an element that is:
related to a feature of the current patient that differs from a corresponding feature in the first prior patient record, and
related to a feature of the current patient that is similar to a corresponding feature in the second prior patient record.

14. The non-transitory computer-readable medium of claim 13, wherein the database comprises hundreds of prior patient records.

15. The non-transitory computer-readable medium of claim 14, wherein each prior patient record comprises at least ten prior patient features that are each assigned a non-binary numerical value.

16. The non-transitory computer-readable medium of claim 13, wherein the composite distance measure is a Mahalanobis distance.

17. The non-transitory computer-readable medium of claim 13, wherein the composite distance measure is one of: a Euclidean distance and a city block distance.

18. The non-transitory computer-readable medium of claim 13, wherein:
each prior patient record is associated with a prior patient,
each prior patient record also includes prior patient information comprising at least one of: clinical information about the previous patient, calculated information about the previous patient, treatment plans of the previous patient, and outcome information of the previous patient,
the current patient record also includes current patient information comprising at least one of: clinical information about the current patient, calculated information about the current patient, treatment plans of the current patient, and outcome information of the current patient, and
the composite distance measure is further based on the current and prior patient information.
